(19) United States
(12) Patent Application Publication
(10) Pub. No.: US 2010/0196920 A1
(43) Pub. Date: Aug. 5, 2010

(54) NANOSCOPIC BIOMOLECULAR ABSORPTION SPECTROSCOPY ENABLED BY SINGLE NANOPARTICLE PLASMON RESONANCE ENERGY TRANSFER

(75) Inventors: Luke P. Lee, Orinda, CA (US); Gang L. Liu, Champaign, IL (US); Yitao Long, Shanghai (CN)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE REGENTS OF THE UNIVERSITY OF CALIFORNIA, Oakland, CA (US)

(21) Appl. No.: 12/599,639

(22) PCT Filed: May 9, 2008
(86) PCT No.: PCT/US2008/005940

§ 371 Date: Apr. 19, 2010

Related U.S. Application Data
(60) Provisional application No. 60/917,211, filed on May 10, 2007.

(51) Int. Cl. G01N 33/53; G01N 33/543

(52) U.S. Cl. 435/7.1; 436/518; 977/773; 977/904

(57) ABSTRACT

The disclosure provides methods and compositions useful for measuring a target analyte in a sample with nanoparticle plasmon resonance. In particular the disclosure provides methods and compositions for measuring a target analyte comprising plasmon resonance energy transfer.

### CROSS REFERENCE TO RELATED APPLICATIONS

This application claims priority under 35 U.S.C. §119 from Provisional Application Ser. No. 60/917,211, filed May 10, 2007, the disclosure of which is incorporated herein by reference.

### TECHNICAL FIELD

The disclosure relates to methods and compositions useful in the detection of molecular agents. More particularly, the compositions and methods of the disclosure related to the use of plasmon resonance energy transfer nanoparticles and methods.

### BACKGROUND

Measurement of an analyte concentration in vitro or in vivo by non-invasive techniques can help elucidate the physiological function of the analyte. This can also aid in identifying changes that occur in a cell or organism in response to physiological stimuli or the presence of absence of an analyte in a sample.

### SUMMARY

The disclosure provides a method for detection of a target analyte, comprising: (a) providing a plurality of nanostructures; (b) contacting the plurality of nanostructures with a fluid suspected of or having the target analyte; (c) contacting the fluid with an electromagnetic radiation at a desired wavelength sufficient to cause plasmon resonance of the nanostructure; and (d) detecting plasmon resonance energy transfer (PRET) from a PRET partner in the fluid, wherein the PRET partner is indicative of the presence of the target analyte.

The disclosure also provides a plasmon resonance indicator comprising: a metallic nanostructure that undergoes resonance when exposed to electromagnetic radiation; and a binding ligand that binds to a mettalo-biomolecule.

The disclosure further provides a Plasmon resonance indicator comprising: a metallic nanostructure functionalized to bind to a metallo-biomolecule.

Composition comprising a Plasmon resonance indicator of the disclosure are also provided herein in combination with a pharmaceutically acceptable carrier.

The disclosure also provides a composition comprising a Plasmon resonance energy indicator linked to a metallo-biomolecule. In one aspect, the nanostructure comprises a metal. In yet another aspect, the metallobiomolecule comprises a metal that undergoes resonance energy emission when the biomolecule is within a resonance distance of the nanostructure.

The disclosure further provides a method of detecting a metallo-biomolecule comprising: exposing the composition above to electromagnetic radiation, wherein the metallic nanostructure and metallo-biomolecule undergo Plasmon resonance energy transfer, and detecting a spectral change when the metallic nanostructure and metallo-biomolecule are within resonance energy distance compared with either the metallo-biomolecule or metallic nanostructure alone. In one embodiment, the distance between a nanostructure and metallo-biomolecule are changed. In yet another embodiment, the distance is changed by cleaving a linking agent linking a nanostructure and metallo-biomolecule.

The disclosure provides a plasmon resonance indicator comprising: a nanoparticle that undergoes plasmon resonance upon exposure to an appropriate electromagnetic radiation, the nanoparticle having an analyte-binding region which binds an analyte an acceptor agent coupled to the analyte, wherein the nanoparticle and the acceptor agent are position relative to each other such that the nanoparticle and analyte undergo plasmon resonance energy transfer when the nanoparticle is contacted with electromagnetic radiation.

The disclosure further comprises a method for determining the concentration of an analyte in a sample comprising: contacting the sample with the plasmon resonance indicator as set forth herein, exciting the nanoparticle; and determining the degree of plasmon resonance energy transfer in the sample corresponding to the concentration of the analyte in the sample.

The disclosure provides a nanostructure that undergoes plasmon resonance energy transfer (PRET) when contacted with electromagnetic radiation. In one embodiment the nanostructure comprise a geometric shell having an opening defined by a sharp edge. In yet another embodiment, the nanostructure comprises one or more noble metals. In yet further embodiment, the nanostructure comprises a functional group that associates with a target analyte.

The disclosure provides a plasmon resonance indicator comprising: a nanoparticle that undergoes plasmon resonance upon exposure to an appropriate electromagnetic radiation having an analyte-binding region which binds an analyte an acceptor agent coupled to the analyte; wherein the nanoparticle and the acceptor agent are position relative to each other that the nanoparticle and analyte undergo plasmon resonance energy transfer (PRET) when the nanoparticle is contacted with electromagnetic radiation.

The disclosure also provides a method for determining the concentration of an analyte in a sample comprising: contacting the sample with the plasmon resonance indicator of the disclosure, exciting the nanoparticle; and determining the degree of plasmon resonance energy transfer in the sample corresponding to the concentration of the analyte in the sample. In one aspect, the step of determining the degree of plasmon resonance energy transfer in the sample comprises measuring light emitted the acceptor agent of the analyte. In another aspect, determining the degree of plasmon resonance energy transfer in the sample comprises measuring light emitted from the nanoparticle, measuring light emitted from the acceptor agent, and calculating a ratio of the light emitted from the nanoparticle and the light emitted from the acceptor agent. In another aspect, the step of determining the degree of plasmon resonance energy transfer in the sample comprises measuring the excited state lifetime of the nanoparticle. The method can further comprise the steps of determining the concentration of the analyte at a first time after contacting the sample with the nanoparticle, determining the concentration of the analyte at a second time after contacting the sample with the nanoparticle, and calculating the difference in the concentration of the analyte at the first time and the second time, whereby the difference in the concentration of the analyte in the sample reflects a change in concentration of the analyte present in the sample. The method can further comprise the step of contacting the sample with a compound between the first time and the second time, whereby a difference in the concentration of the analyte in the sample between the first time and the second time indicates that the compound alters the presence of the analyte. In another aspect, the sample comprises an intact cell and the contacting step.

The disclosure provides a nanostructure that undergoes plasmon resonance energy transfer (PRET) when contacted with electromagnetic radiation. In one embodiment, the nanostructure a geometric shell having an opening defined by a sharp edge.

The disclosure also provides a pharmaceutical composition comprising a plurality of nanostructures of the disclosure in a pharmaceutically acceptable carrier.

The disclosure provides a plasmon resonance indicator of the disclosure comprising a functional group that associates with a target analyte.

The disclosure also provides a method for detection of a target analyte, comprising: a) providing a plurality of nanostructures of the disclosure; b) a device that measures emitted electromagnetic radiation; c) contacting the plurality of nanostructures with a fluid suspected of or having the target analyte; d) contacting the fluid with an electromagnetic radiation at a desired wavelength sufficient to cause plasmon resonance of the nanoparticle; and e) detecting plasmon resonance energy transfer from the using the device.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A-D shows a schematic diagram of PRET enabled nanoscopic biomolecular absorption spectroscopy. (A) PRET from a single metallic nanoparticle to surface conjugated biomolecules. The wavelength-specific plasmon resonance (collective free electron oscillation) in metallic nanoparticle is excited by white light illumination. The plasmon resonance dipole can interact with the biomolecular dipole and transfer energy to biomolecules. (B) Hybrid energy diagram showing quantized energy transfer process. With optical excitation the free electrons in the conduction band of metallic nanoparticle are elevated from Fermi to higher energy level forming resonating plasmon. The plasmon resonance energy is transferred to biomolecule comprising a moiety with an absorption spectrum capable of absorbing the resonance energy of the nanoparticle (e.g., here depicted as a metalloprotein biomolecules such as Cyt c) conjugated on the nanoparticle surface when matched with the electronic transition energy in biomolecule optical absorption. (C) Principle Rayleigh scattering spectrum of the single PRET probe in (A). The energy transition in PRET is represented as quenching dips in nanoparticle scattering spectrum, and the dip positions correspond to the biomolecule optical absorption peaks. (D) Experimental system configuration. The biomolecule conjugated nanoparticles are immobilized and covalently tethered on the glass surface and immersed in buffer solutions. The white light is illuminated on individual nanoparticles at oblique angles by a darkfield condenser lens. The forward scattering light from the nanoparticles is collected by a microscopy objective lens, imaged by a true color camera, and analyzed by a spectrograph system. The pictures shown are (left) the true color nanoparticles scattering image and (right) the zero-order spectrograph image of a few nanoparticles selected by the entrance slit of the spectrophotometer.

FIG. 2A-F shows experimental results of PRET from single gold nanoparticle to conjugated Cyt c molecules. The Rayleigh scattering spectrum of a single gold nanoparticle coated with (A) only Cysteamine coating, (B) Cysteamine cross linker and reduced Cyt c and (C) Cyteamine and oxidized Cyt c. The Rayleigh scattering spectrum was obtained using 1 sec integration time. (D) The visible absorption spectra of Cyt c bulk solution in reduction form (blue solid line), and in oxidation form (red solid line) measured in conventional UV-vis absorption spectroscopy. (E) The fitting curve for the spectrum in (B). Black open circle: raw data, Green solid line: fitting curve of the raw data, Yellow solid line: Lorenzian scattering curve of bare gold nanoparticle, Red solid line: Differential absorption spectra for the reduced conjugated Cyt c by subtracting yellow curve from the green curve. (F) The fitting curve for the spectrum in (B). Black open circle: raw data, Green solid line: fitting curve of the raw data, Yellow solid line: Lorenzian scattering curve of bare gold nanoparticle, Blue solid line: Differential absorption spectra for the oxidized conjugated Cyt c by subtracting yellow curve from the green curve.

FIG. 3 A-C shows negative control results showing the energy matching for PRET. (A) The scattering spectrum spectra of a 30 nm gold nanoparticle coated with Cys-(Gly-Hyp-Pro)⁶ peptides. (B) The scattering spectrum of a single large gold nanoparticle cluster conjugated with Cysteamine and Cyt c. (C) The scattering spectrum of a 40 nm amine-modified polystyrene bead conjugated with Cyt c.

FIG. 4A-B is a simulation of nanoparticle plasmon resonance coupling to a single Cyt c molecule. (A) Time averaged total electromagnetic (EM) energy at 550 nm polarized light excitation around the interface of a single 30 nm gold nanoparticle and a single 3 nm spherical molecules. The dielectric nanosphere is used to simulate single reduced Cyt c molecule with a wavelength-dependent complex refractive index. The EM energy is transferred to the single molecule and forms the dipolar energy distribution across the molecule. The inset image of the whole nanoparticle shows the energy coupling only occurs in the light polarization direction. (B) Time averaged total EM energy profile at the cross sectional line in (A) as the function of the excitation wavelength or energy. The EM energy distribution at each wavelength is normalized to the average EM energy inside the nanoparticle. The representative line plots of the energy profile at 370 nm, 550 nm and 730 nm are superposed on the 2D color-coded energy distribution at corresponding wavelength positions. The EM energy of the nanoparticle is coupled to the single biomolecule around 550 nm forming a dipolar energy distribution across the biomolecule, while at other wavelengths much less energy transfer is observed.

FIG. 5A-B shows scattering spectra. (A) Raw scattering spectra of four representative gold nanoparticles conjugated with reduced Cyt c molecules. The nanoparticle plasmon resonance peaks and PRET-induced plasmon quenching dips have variable intensities from particle to particle due to the non-uniformity of the conjugated molecule number and particle geometry; however the plasmon quenching peak positions are consistent. More than 50 individual nanoparticles were tested and PRET can be consistently observed. (B) Time-lapse measurement of scattering spectra of a single gold nanoparticle conjugated with reduced Cyt c molecules. The plasmon quenching spectral dips remain nearly constant during the whole time period of measurement. No photobleaching effect was observed.

FIG. 6A-B is a simulation of electromagnetic (EM) energy in the single Cyt c molecule in FIG. 4 as the function of nanoparticle size and material. (A) Normalized average EM energy in single Cyt c molecules as the function of wavelength for different sizes of gold nanoparticles. The average EM energy in Cyt c is normalized to the average EM energy in gold nanoparticles. (B) Normalized average EM energy in single Cyt c molecule at the wavelength of 550 nm as the function of nanoparticle size and material.

FIG. 7A-C shows the PRET spectra for 3 representative gold nanoparticles conjugated with reduced cytochrome c molecules. The nanoparticle plasmon resonance wavelengths and the intensities of PRET-induced plasmon quenching dips vary from particle to particle in (a)-(c); however the plasmon quenching peak positions are consistent. Open circle: raw data, with fitting curve, top solid line: Lorentzian scattering curve of bare gold nanoparticle, bottom solid line: processed absorption spectra for the reduced conjugated cytochrome c by subtracting red curve from the green curve. The scale bars in the inset images stand for 2 μm.

### DETAILED DESCRIPTION

As used herein and in the appended claims, the singular forms “a,” “and,” and “the” include plural referents unless the context clearly dictates otherwise. Thus, for example, reference to “a nanoparticle” includes a plurality of such nanoparticle and reference to “the analyte” includes reference to one or more analytes known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The signature of a noble metal nanostructure is the localized surface plasmon resonance. This resonance occurs when the correct wavelength of electromagnetic energy (e.g., light) strikes a noble metal nanostructure causing the plasma of conduction electrons to oscillate collectively. The resonance oscillation is localized near the surface region of the nanostructure. Such resonance is advantageous in that the nanostructure is selectively excited at a particular photon absorption, which results in the generation of locally enhanced or amplified electromagnetic fields at the nanostructure surface. The resonance for noble metal nanostructures (e.g., in the 20-500 nm range) occurs in the visible and IR regions of the spectrum and can be measured by UV-visible-IR extinction spectroscopy. In some nanostructures, the nanostructure can be tuned to generate a particular absorbance and emission spectra by adjusting the metallic composition and geometry.

Free electrons in the conduction band of metallic nanostructure (e.g., a nanoparticle) can be excited by an external optical field to form a collective electron oscillation called nanoparticle plasmon resonance. Distinctive from the propagating surface plasmon resonance on metallic thin film, plasmon resonance energy in a nanoparticle is spatially confined within the physical boundary of the nanoparticle. It has been shown that confined plasmon resonance energy in a single metallic nanoparticle can be continuously transferred to adjacent metallic nanoparticles in the same material and size through plasmon coupling.

A Plasmon resonance indicator takes advantage of the emission spectra produced by resonance of a nanostructure for excitation of nearby or surrounding acceptor resonance analytes. The Plasmon resonance indicator comprises either a nanostructure or a metallo-molecular entity or a combination (e.g., a pair) that cause plasmon resonance energy transfer (“PRET”). The spectral signature produced by such PRET pairs provides the ability to measure the presence of concentration of an analyte on the nanoscale. During use the nanostructure is capable of excitation by an energy wavelength resulting in an emission spectra by the nanostructure. The emission spectra is capable of excitation of an acceptor moiety either alone or as part of a larger molecule (e.g., an analyte or a moiety of the analyte). The acceptor moiety in-turn emits a detectable emissions spectra. The donor and acceptor agents can be chosen such that the excitation spectrum of one of the agents (the acceptor agent) overlaps with the emission spectrum of the excited nanostructure (the donor agent).

Any nanostructure capable of plasmon resonance can be used in the disclosure. In addition, any number of acceptor agents can be used wherein the acceptor agent is capable of excitation-emission.

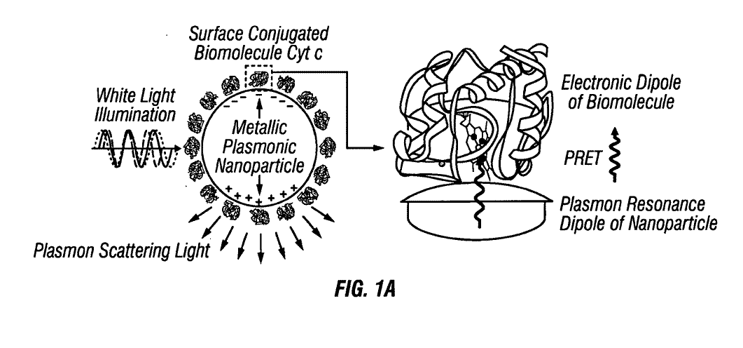

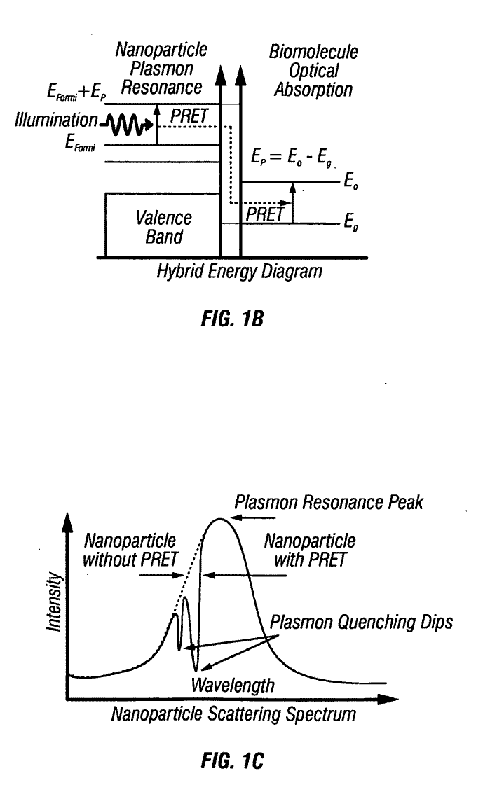

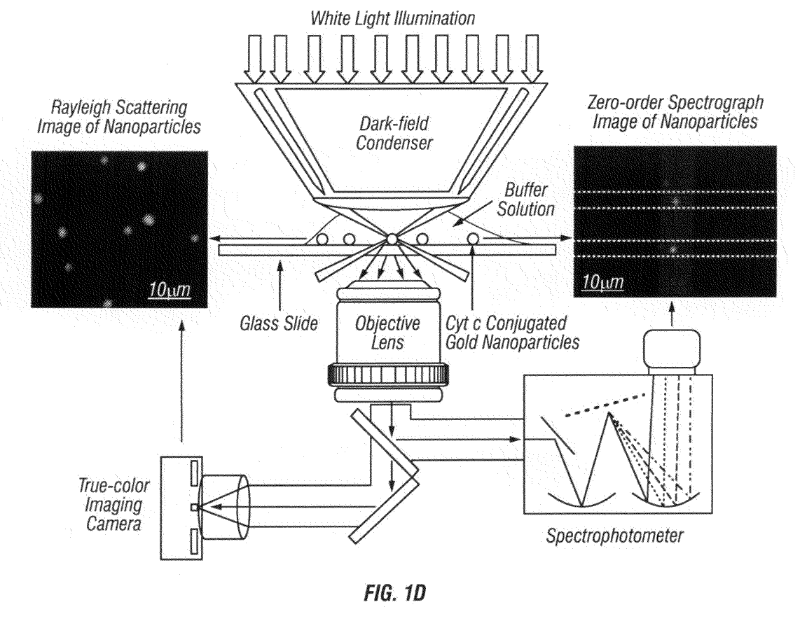

Referring to FIG. 1, the donor nanostructure (e.g., the nanoparticle) is linked to an analyte comprising an acceptor moiety (typically an agent comprising a metal) such that the donor and acceptor are operatively linked or within resonance excitation distance and can undergo PRET. The presence of PRET (e.g., either an emission spectra or a quenching spectra) is indicative of the presence of the analyte. Alternatively, the dissociation of an analyte comprising an acceptor agent from a donor nanostructure is a measurable event wherein the decrease or absence of PRET is indicative of a dissociation event (e.g., enzyme cleavage, oxidation or reduction of a metal and the like). The donor nanostructure (e.g., the nanoparticle) is excited by a wavelength (e.g., light) of appropriate intensity within the excitation spectrum of the donor nanostructure (e.g., nanoparticle) (λ_{excitation}) The donor nanostructure emits the absorbed energy as emission energy, e.g., light (λₑₘᵢₛₛᵢₒₙ₁). When the acceptor is positioned to quench the donor nanostructure in the excited state, the energy is transferred to the acceptor which can emit its own resonance spectra (λₑₘᵢₛₛᵢₒₙ₂). PRET can be manifested as a reduction in the intensity of the signal from the donor (λₑₘᵢₛₛᵢₒₙ₁), reduction in the lifetime of the excited state of the donor, or emission of a resonance spectra at different wavelength (lower energies) characteristic of the acceptor agent (λₑₘᵢₛₛᵢₒₙ₂). Accordingly, PRET is increased (or decreased) depending upon the distance between the donor nanostructure and the acceptor agent.

As used herein a “donor” generally refers to the molecular entity (e.g., the nanostructure) whose resonance is generated by an external incident wavelength. An “acceptor” refers generally to an molecular entity whose resonance is generated by donor entity.

The disclosure demonstrates nanoscopic absorption spectroscopy enabled by plasmon resonance energy transfer (PRET) from a single metallic nanoparticle to a biomolecule conjugated on the surface of a nanoparticle. Furthermore, the disclosure provides methods and compositions useful for PRET measurements.

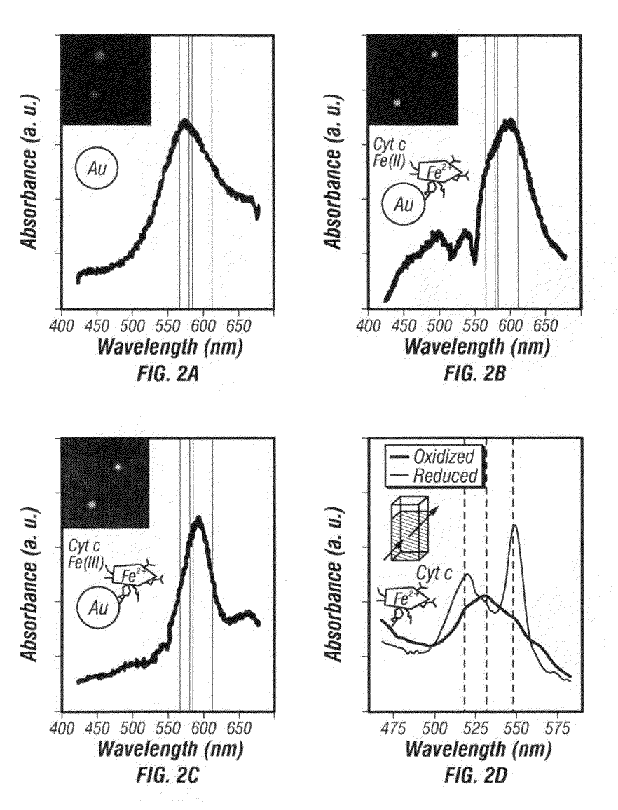

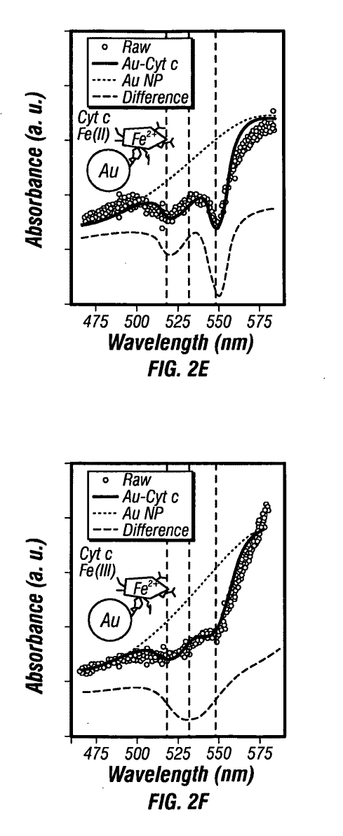

The plasmon resonance of gold and silver nanoparticles conjugated with various biomolecules such as DNA, peptide, biotin-streptavidin has been studied by single particle Rayleigh scattering spectroscopy. These studies demonstrated the shift of plasmon resonant wavelength by changing dielectric medium due to structural changes of a biomolecule conjugated on the surface of single metallic nanoparticles. Since most of the cases have conjugated biomolecules with optical absorption peaks in ultraviolet (UV) or far infrared range on gold and silver nanoparticles with visible plasmon resonance peaks, only the shift of plasmon resonance peak was observed. The disclosure demonstrates that a conjugated biomolecule on a nanoparticle provides a measurable Plasmon resonance energy transfer from the nanoparticle to the conjugated biomolecule. For example, a conjugated metalloprotein, Cytochrome c (Cyt c), on a single 30 nm gold nanoparticle provides measurable plasmon resonance energy transfer from the nanoplasmonic particle to Cyt c (FIG. 1A and FIG. 2). The intentional overlap of the absorption peak positions of desired biomolecules with the plasmon resonance peak of the metallic nanoparticle generates distinguishable spectral dips on the Rayleigh scattering spectrum of a single nanoparticle, which also allows near single molecular level nanoscopic absorption spectroscopy (FIGS. 1 and 2).

Any number of biomolecules capable of undergoing plasmon energy transfer can be used (e.g., small molecule chemicals, nucleic acids, proteins, peptides and lipids). For example, biomolecules capable of undergoing PRET include metalloproteins, metallopeptides, metallopolypeptides and the like. A metallo-protein, -polypeptide, or -peptide generally refers to protein, polypeptide or peptide that contains a metal cofactor. The metal may be an isolated ion or may be coordinated with a nonprotein organic compound, such as the porphyrin found in hemoproteins. In some biomolecules, the metal is co-coordinated with a side chain of the protein and an inorganic nonmetallic ion. This kind of protein-metal-nonmetal structure is typically found in iron-sulfur clusters.

A group of metalloproteins includes the metalloenzymes. Metalloenzymes typically contain one or more metal atoms as functional parts of their structures. These metals are often involved in enzyme catalysis, such as in carbonic anhydrase, cytochrome P450's and cytochrome c oxidase. Metal ions usually form part of the active site as they can be multicoordinated and thus held in a protein while having a high affinity for the substrate through a lone pair.

Metalloproteins, polypeptides and peptides can also include artificially generated (or recombinant) polypeptides comprising a metal-containing moiety. For example, Cupric containing proteins include: Cytochrome oxidase, Superoxide dismutase; Ferrous or Ferric containing proteins include: Catalase, Cytochrome(via Heme), Nitrogenase, and Hydrogenase; Magnesium containing proteins include: Glucose 6-phosphatase and Hexokinase; Manganese containing proteins include: Arginase; Molybdenum containing proteins include Nitrate reductase; Nickel containing proteins include: Urease; Selenium containing proteins include: Glutathione peroxidase; Zinc containing proteins include: Alcohol dehydrogenase, Carbonic anhydrase and DNA polymerase.

Furthermore, recombinant proteins comprising a metal ion containing moiety can be developed using skill available in the art (see, e.g., U.S. Application publication no. 20050090649 to Lombardi et al.). Using such techniques binding ligands comprising a metal containing moiety can be developed. Using such techniques, for example, a ligand can be conjugated to a nanoparticle, wherein the ligand does not normally have a metal ion associated with it. A binding partner can be designed using known amino acid sequences or structure, wherein the binding partner is constructed to be linked to or contain a metal ion. Upon binding between the ligand and binding partner the distance between the nanoparticle is sufficient to undergo PRET and thereby detection. Furthermore, a change in oxidation of the metal ion can also be detected. Various metalloprotein can be identified or designed using techniques and databases in the art. Additionally there are a number of non-proteinacious metallo-biomolecules that can be detected and used in combination with the methods and compositions of the disclosure.

A metallo-biomolecule refers generally to a polypeptide, peptide, protein, enzyme, lipid, hormone, nucleic acid of other biological organic factor or metabolite that contains a metal cofactor. The metal may be an isolated ion or may be coordinated with a nonprotein organic compound. In some cases, the metal is co-coordinated with a side chain of the protein, lipid or other organic molecule and an inorganic nonmetallic ion.

The examples provided herein utilize cytochrome c, however, it will be recognized that any metallo-biomolecule (whether natural generated by the hand of man) can be used with the methods and compositions described herein. Cytochrome c (Cyt c), a metalloprotein in cellular mitochondria membrane, acts as the charge transfer mediator and plays a role in bioenergy generation, metabolism, and cell apoptosis. Cyt c has several optical absorption peaks in visible range around 550 nm coinciding with the 30 nm gold nanoparticle plasmon resonance, and more importantly it is a natural energy acceptor with electron tunneling channels. Similar to the donor-acceptor energy matching in Fluorescent (or Förster) Resonance Energy Transfer (FRET) between two fluorophores, the matching of the localized resonating plasmon kinetic energy Ep in gold nanoparticles with the electron transition energy from ground to excited state Ee-Eg in Cyt c molecules permits the PRET process (FIG. 1B). The quantized energy is transferred through the dipole-dipole interaction between the artificial alternating dipole—resonating plasmon in nanoparticle and the biomolecular dipole. The plasmon energy quenching of nanoparticle due to PRET is represented as the “spectral dips” in the single nanoparticle scattering spectrum (FIG. 1C and FIG. 2) and the positions of dips match with the molecular absorption peak positions (FIG. 1C and FIG. 2). PRET is a direct energy transfer process and thus much more efficient and faster than optical energy absorption, so the absorption spectral peaks of conjugated Cyt c molecules on single nanoparticles can be detected with a simple optical system, which is otherwise impossible using conventional visible absorption spectroscopic methods. Although the near field optical excitation efficiency from the nanoparticle scattering light is much higher than far field optical excitation (i.e., the photon scattered from the nanoparticle is more likely to transmit through and be absorbed by the surface conjugated biomolecules than those far away from the nanoparticle), the optical absorption at 550 nm by the ferrocytochrome c molecule monolayer only accounts for 0.03% of the nanoparticle scattering light even for 100% excitation efficiency; therefore, the dramatic spectral dips are not a result of the direct optical absorption of Cyt c molecules.

The metallic composition of composite nanostructures of the disclosure are biocompatible, and thus can be biofunctionalized and applied in real-time biomolecular imaging. Unlike conventional fluorescence imaging, PRET acquires unique signatures of chemical and biological molecules without labeling with fluorophore molecules.

The nanoparticles can be functionalized to selectively interact with a particular target analyte. A target analyte refers to (a) the biomolecule to be detected or (b) a molecule the co-localizes, binds to or associates with a biomolecule to be detected. Accordingly, the target analyte can comprise a metallo-biomolecule or a binding partner of a metallo-biomolecule. In one aspect, the target analyte comprises an acceptor agent (e.g., a metallo-biolmolecule). Attached functional groups can comprise components for specifically, but reversibly or irreversibly, interacting with the specific analyte (e.g., can be labeled for site/molecule directed interactions). For example, a surface bound functional group (e.g., a targeting ligand) can be attached to a nanostructure of the disclosure. For example, a chemical molecule can be immobilized on the surfaces of a nanostructure of the disclosure. For example, a thiol-group containing molecule, can be attached to the surface of the nanostructure through Au sulfide bonds by spreading and drying a droplet of 1.M MTMO in anhydrous ethanol solution.

A targeting ligand can include a receptor bound to the surface of a nanostructure of the disclosure that interacts reversibly or irreversibly with a target analyte or specific metallo-biomolecule (e.g., a metalloprotein and the like). Typically, the interaction of the targeting ligand and the analyte lasts sufficiently long for detection of the metallo-biomolecule by PRET. The targeting ligand attached to the nanoparticle is used to colocalize the nanoparticle (e.g., the donor moiety) with a biomolecule comprising an acceptor moiety (e.g., a metal) that serves to form a PRET pair.

Examples of targeting ligands that can be linked to a nanoparticle include antigen-antibody pairs, receptor-ligand pairs, and carbohydrates and their binding partners. The targeting ligand may be nucleic acid, when nucleic acid binding proteins are the targets. As will be appreciated by those in the art, the composition of the targeting ligand will depend on the composition of the target analyte. Binding ligands to a wide variety of analytes are known or can be readily identified using known techniques. A target analyte can be the metallo-biomolecule itself, or a molecule that co-localizes to a metallo-biomolecule.

For example, when the metallo-biomolecule is a single-stranded nucleic acid, the binding/targeting ligand is generally a substantially complementary nucleic acid. Similarly, when the metallo-biomolecule is a nucleic acid binding protein (e.g., a metallo-protein) the capture binding ligand is either a single-stranded or double-stranded nucleic acid or a binding agent capable of binding with the metallo-protein (e.g., an antibody or nucleic acid oligonucleotide). When the target analyte is a protein, the binding ligands include proteins or small molecules. For example, when the target analyte is an enzyme, suitable binding ligands include substrates, inhibitors, and other proteins that bind the enzyme, i.e. components of a multi-enzyme (or protein) complex. As will be appreciated by those in the art, any two molecules that will associate, may be used, either as the target analyte or the functional group (e.g., targeting/binding ligand). Suitable analyte/binding ligand pairs include, but are not limited to, antibodies/antigens, receptors/ligand, proteins/nucleic acids; nucleic acids/nucleic acids, enzymes/substrates and/or inhibitors, carbohydrates (including glycoproteins and glycolipids)/lectins, carbohydrates and other binding partners, proteins/proteins; and protein/small molecules. In one embodiment, the binding ligands are portions (e.g., the extracellular portions) of cell surface receptors. In one embodiment one or both of the binding pairs comprises a moiety that completes a PRET pair, wherein one member of the PRET pair comprises a nanoparticle. In yet another embodiment, one or both of the binding ligand pairs comprises a metal moiety.

Target analytes that can be detected or measured by the compositions and methods of the disclosure include any molecule or atom or molecular complex suitable for detection by the nanostructures of the disclosure. Examples of such analytes include, but are not limited to, biomolecules such as proteins, peptides, polynucleotides, lipids and the like, glucose, ascorbate, lactic acid, urea, pesticides, chemical warfare agents, pollutants, and explosives.

In some embodiments, the disclosure provides kits and systems for use in monitoring the level of an analyte in a sample or subject. In some embodiments, the kits are for home use by a subject to assist in identifying an analyte, disease or disorder or to monitor a biological condition. For example, in some embodiments, a sensor is delivered to the subject (e.g., by a medical professional) and the subject is provided with a device for monitoring levels of an analyte (e.g., the subject places the device near the nanostructure location or suspected location and the device provides a reading of the level of the analyte).

The disclosure has use in the detection of analytes in the environment, including explosive and biological agents. Accordingly, the disclosure is useful in Homeland Security and the military for detection of analytes. In one embodiment, the disclosure provides kits for monitoring military personnel in a war situation where they may be exposed to toxins. The nanostructures are administered or contacted with the subject prior to potential exposure. The subjects can then be monitored at set intervals using a detection device.

Excitation of the nanostructures of the disclosure is performed by contacting the nanostructure with appropriate electromagnetic radiation (e.g., an excitation wavelength). Wavelengths in the visible spectrum comprise light radiation that contains wavelengths from approximately 360 nm to approximately 800 nm. Ultraviolet radiation comprises wavelengths less than that of visible light, but greater than that of X-rays, and the term “infrared spectrum” refers to radiation with wavelengths of greater 800 nm. Typically, the desired wavelength can be provided through standard laser and electromagnetic radiation techniques.

The nanostructures of the disclosure can be used in vivo and in vitro to detect, identify, and/or characterize analytes of interest. The nanostructures can be used to detect analytes in environmental samples as well as samples derived from living organisms. As used herein, the term “sample” is used in its broadest sense. For example, a sample can comprise a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. The nanostructures can be used, for example, in bodily fluids in vivo or in vitro. Such bodily fluids include, but are not limited to, blood, serum, lymph, cerebral spinal fluid, aqueous humor, interstitial fluid, and urine.

Commercial applications include environmental toxicology, materials quality control, food and agricultural products monitoring, anesthetic detection, automobile oil or radiator fluid monitoring, hazardous spill identification, medical diagnostics, detection and classification of bacteria and microorganisms both in vitro and in vivo for biomedical uses and medical diagnostic uses, infectious disease detection, body fluids analysis, drug discovery, telesurgery, illegal substance detection and identification, and the like.

A number of devices can be used for measuring plasmon resonance energy. Any device suitable for detection of a signal from the nanostructure of the disclosure at wavelengths from the non-visible, visible and infrared. In some embodiments, the device includes delivery and collection optics, a laser source, a notch filter, and detector.

A nanoparticle is a particle having one or more dimensions of the order of 100 nm or less. The geometry of the particle is not critical. For example, the nanoparticle can be in the shape of a sphere, half moon, bowl, rod and the like.

In PRET, the “donor agent” and the “acceptor agent” are selected so that the donor and acceptor agents exhibit resonance energy transfer when the donor agent is excited. One factor to be considered in choosing the donor/acceptor pair is the efficiency of PRET between the two agents. Typically, the efficiency of PRET between the donor and acceptor agents is at least 10%, commonly at least 50%, and most commonly at least 80%. The efficiency of PRET can be tested empirically using the methods described herein and known in the art, particularly, using the conditions set forth in the Examples.

“Analyte” refers to a molecule (e.g., a polypeptide, peptide, polynucleotide, small molecule, ligand, receptor, enzyme and the like).

“Operatively linked” refers to a juxtaposition wherein the components so described are in a relationship permitting them to function in their intended manner. For example, a nanoparticle is operatively linked to an analyte if the nanoparticle is in an association by bond formation (e.g., covalently, through a ligand, or through non-covalent interactions) with an analyte of interest such that the nanoparticle is capable of resonating upon exposure to an excitation energy.

The efficiency of PRET depends on the separation distance and the orientation of the donor and acceptor Plasmon resonance agents. The characteristic distance R₀ at which PRET is 50% efficient depends on the quantum yield of the donor agent (i.e., the shorter-wavelength), the extinction coefficient of the acceptor agent (i.e., the longer-wavelength), and the overlap between the emission spectrum of the donor agent and the excitation spectrum of the acceptor agent. R₀ is given (in Å).

These factors need to be balanced to optimize the efficiency and detectability of PRET. The emission spectrum of the donor nanoparticle agent should overlap as much as possible with the excitation spectrum of the acceptor agent (e.g., an analyte comprising a metal). In addition, the excitation spectra of the donor and acceptor agents should overlap as little as possible so that a wavelength region can be found at which the donor agent can be excited selectively and efficiently without directly exciting the acceptor agent. Direct excitation of the acceptor agent should be avoided. Similarly, the emission spectra of the donor and acceptor agents should have minimal overlap so that the two emissions can be distinguished.

The amount of analyte in a sample can be determined by determining the degree of PRET in the sample. Changes in analyte concentration can be determined by monitoring PRET at a first and second time. The amount of analyte in the sample can be calculated by using a calibration curve established by titration.

A nanostructure of the disclosure can be formulated with a pharmaceutically acceptable carrier, although the nanostructure may be administered alone, as a pharmaceutical composition. The nanostructures are useful for measuring the concentration, presence or change of a target analyte in vivo or in vitro. Appropriate carriers and delivery methods are known in the art as described more fully herein.

A pharmaceutical composition according to the disclosure can be prepared to include a nanostructure of the disclosure, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The pharmaceutical compositions according to the disclosure may be administered locally or systemically. By “effective dose” is meant the quantity of a nanostructure according to the disclosure to sufficiently provide measurable PRET. Amounts effective for this use will, of course, depend on the tissue and tissue depth, route of delivery and the like.

Typically, dosages used in vitro may provide useful guidance in the amounts useful for administration of the pharmaceutical composition, and animal models may be used to determine effective dosages for specific in vivo techniques. Various considerations are described, e.g., in Langer, Science, 249: 1527, (1990); Gilman et al. (eds.) (1990), each of which is herein incorporated by reference.

As used herein, “administering an effective amount” is intended to include methods of giving or applying a pharmaceutical composition of the disclosure to a subject that allow the composition to perform its intended function.

The pharmaceutical composition can be administered in a convenient manner, such as by injection (e.g., subcutaneous, intravenous, and the like), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit.

For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Supplementary active compounds can also be incorporated into the compositions.

The following examples are intended to illustrate but not limit the disclosure. While they are typical of those that might be used, other procedures known to those skilled in the art may alternatively be used.

### EXAMPLES

As shown in the experimental configuration (FIG. 1D), the Cyt c conjugated 30 nm gold nanoparticles are dispersedly tethered on the surface of a transparent glass slide. The glass slide is mounted on a white light darkfield microscopy system with a true-color camera and a spectrometer to characterize the scattering image and spectrum of individual gold nanoparticles as well as the hybrid PRET probes (i.e. specific metallic nanoparticles with conjugated Cyt c molecules).

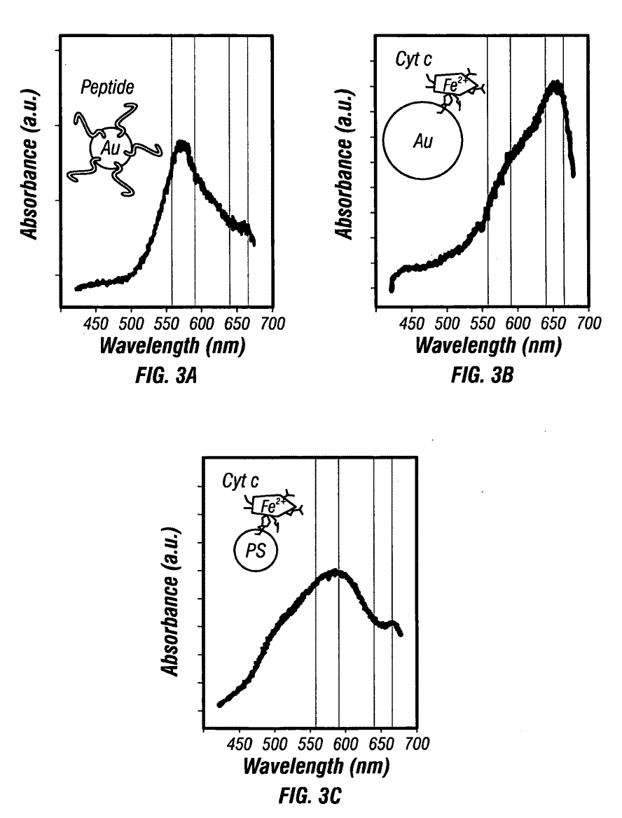

In comparison with the visible scattering spectrum of gold nanoparticles coated with only Cysteamine cross linker molecules (FIG. 2A), the raw scattering spectra of gold nanoparticles conjugated with reduced (FIG. 2B) and oxidized Cyt c (FIG. 2C) show not only a scattering peak (plasmon resonance peak) but distinctive dips next to it. The spectral dips modulated on the nanoparticle scattering spectrum can be decoupled and converted to the visible absorption peaks of the reduced and oxidized Cyt c molecules (FIGS. 2E and 2F). In accordance with the conventional visible absorption spectrum of bulk Cyt c solutions (FIG. 2D), the processed spectra have matched absorption peaks of reduced Cyt c around 525 nm and 550 nm, and oxidized Cyt c at 530 nm. The energy matching condition in PRET is further confirmed by three negative control experiments. For the first control experiment, synthesized peptides which have absorption peaks out of wavelength range of the plasmon resonance of a 30 nm gold nanoparticle are intentionally conjugated. As expected the scattering spectrum of this hybrid system shows only the scattering peak because the absorption peaks of peptide do not coincide with the plasmon resonance spectrum of the nanoparticle (FIG. 3A). For the second control experiment, the importance of matching resonant frequency and molecular absorption peaks was tested by using a large gold nanoparticle cluster which has a plasmon resonance wavelength beyond 650 nm. As anticipated, the conjugated Cyt c absorption peaks can be hardly observed, including the 525 nm and 550 nm peaks for reduced Cyt c (FIG. 3B). For the third control experiment, dielectric polystyrene nanoparticles were conjugated with Cyt c and characterized. As expected, the plasmon quenching spectral dips cannot be found on the scattering spectrum of single dielectric polystyrene nanoparticle without plasmon resonance even though the dielectric nanoparticle also scatters light, which indicates the presence of excited free electrons is necessary for the PRET process (FIG. 3C).

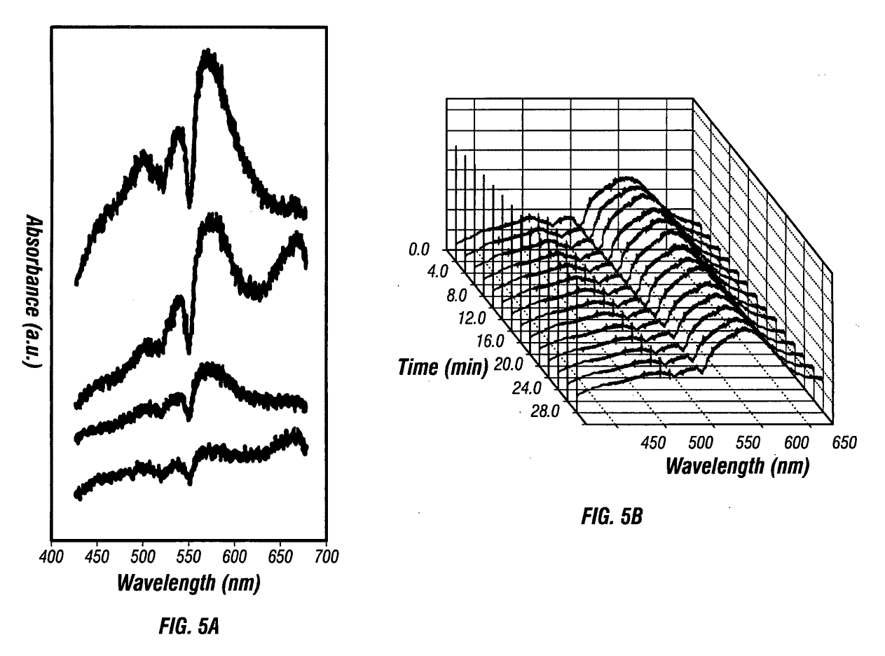

The average surface density of Cyt c molecules on an individual gold nanoparticle is controlled by the molar concentration ratio used in the conjugation process. Considering the effective cross sectional area of single Cyt c molecules and the surface area of single 30 nm gold nanoparticle, maximally around 400 Cyt c molecules can be tethered on a 30 nm gold nanoparticle. The scattering spectrum of many individual 30 nm nanoparticles were measured and extracted the reduced Cyt c visible absorption peaks. Due to the non-uniformity of the surface molecule numbers on each particle and nanoparticle variations, the Cyt c absorption peak intensity shows variations from particle to particle (FIG. 5A); whereas the spectral measurement on each individual nanoparticle is repeatable and stable (FIG. 5B), and no photochemical changes are observed. Unpolarized white light source is used in all the above experiments.

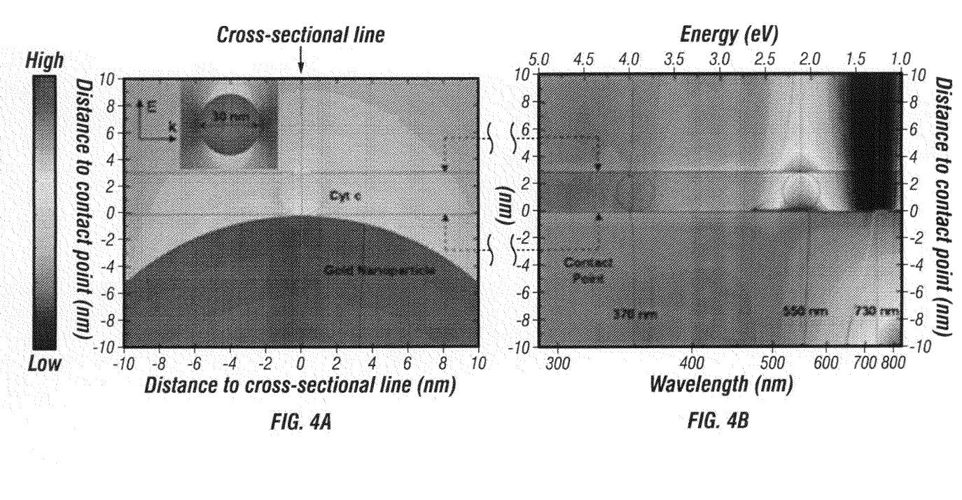

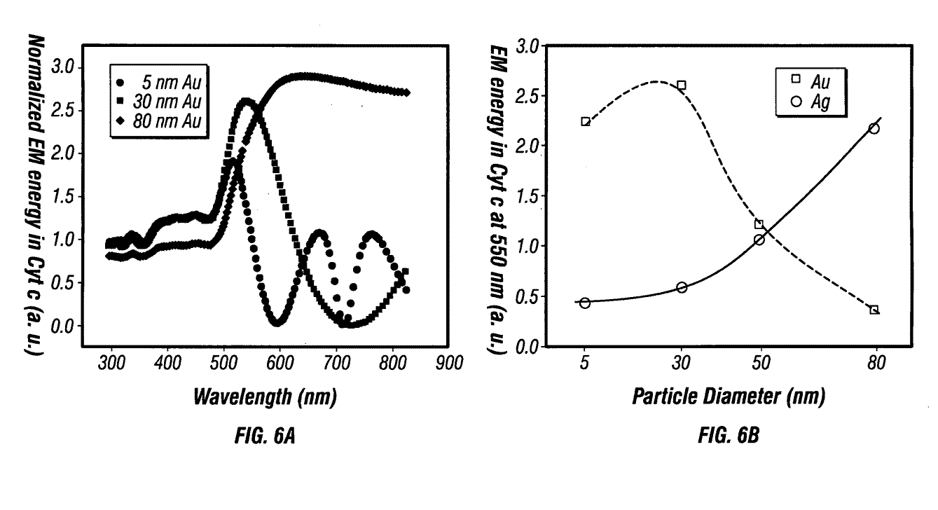

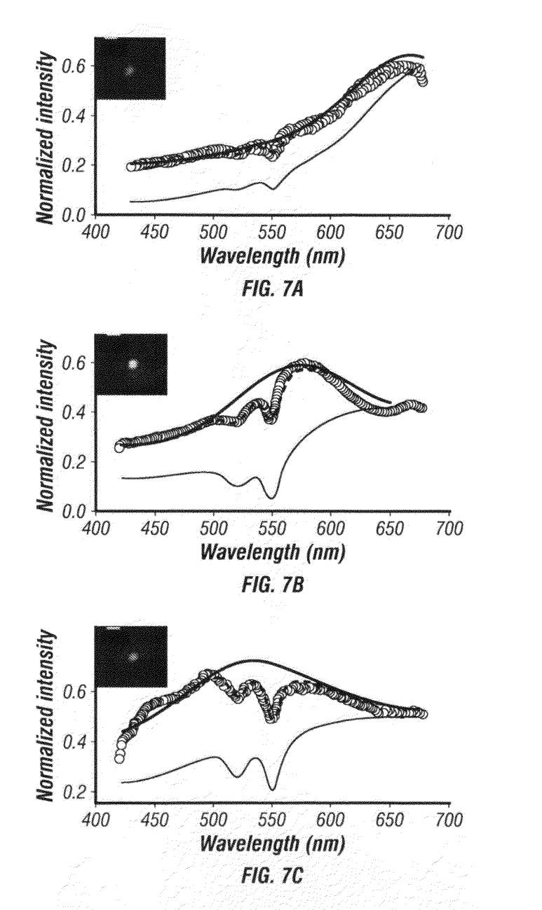

Similar to the energy transfer process in PRET, the PRET efficiency is dependent on the distance from the spectrally active agent of biomolecules, e.g., Heme group for Cyt c, to the plasmonic nanoparticle surface as well as the relative orientations between the polarized plasmon resonance dipole and molecular dipoles (FIG. 4A). On the other hand, the simulated single nanoparticle PRET spectra (FIG. 4B) shows that the strongest plasmon resonance mode for a single Cyt c conjugated 30 nm gold nanoparticle occurs around 550 nm (FIG. 6A). This resonant frequency (i.e. energy) matching condition explains why the plasmon quenching peak amplitude at 550 nm is relatively higher than at 525 nm (FIG. 2E) compared to the peak intensity ratio in Cyt c bulk solution absorption measurement (FIG. 2D). For the nanoparticles of other size and material such as silver, the plasmon resonance energy transferred at 550 nm is less than for 30 nm gold nanoparticles (FIG. 6B) corresponding to the control experimental data (FIG. 3B). The scattering peak wavelength of the gold nanoparticle in experiments is higher than the simulated results due to larger numbers of Cyt c and Cysteamine molecules conjugated on surface.

Although only the PRET in the visible wavelength range is observed here due to the optical properties of gold nanoparticles and Cyt c molecules, the PRET process at UV and near infrared range could be envisioned by using different properties (i.e. size, shape, free electron density, and the like) of metallic nanoparticles with UV or near infrared plasmon resonance wavelength. The PRET-based ultrasensitive biomolecular absorption spectroscopy on single metallic nanoparticle could be used for molecular imaging such as genetic analysis of small copies of latent nucleotides, activity measurements of small numbers of functional cancer biomarker proteins, and rapid detection of little biological toxin, pathogen and virus molecules. Additionally PRET could be applied in intracellular biomolecule conjugated nanoparticle sensors to detect localized in vivo electron transfer, oxygen concentration and pH value changes in living cells with nanoscale spatial resolutions. Furthermore the optical energy in advanced plasmonic devices can be tuned by functional biomolecules taking advantage of the PRET process.

Preparation of Cyt c conjugated gold nanoparticles on glass slide. A cleaned glass slide was modified with 3-mercaptopropyl trimethoxy silane (MTS) by incubation in 1 mM MTS acetone for 24 hours. The glass slide was then rinsed with acetone, dried with clean nitrogen gas. 30 nm spherical gold nanoparticles (Ted Pella, Inc., Redding, Calif.) were cast on and wet the MTS functionalized glass surface. The gold nanoparticles were then immobilized on the by the free thiol groups. The surface was then incubated in 0.1 mM Cysteamine solution for 2 hours. The resulting glass slide was thoroughly rinsed with PBS buffer to remove physically adsorbed Cysteamine, and then incubated in 10 μM horse heart Cyt c PBS solution (pH=7.2) (Sigma, St. Louis, Mo.) for 40 min. Cysteamine has a thiol group at one end to connect with gold and an amino group at other end to anchor the carboxyl groups in the peptide chain of cyt c. The Cyt c molecules are in the oxidized form when purchased, and the reduced form of Cyt c is made by the addition of excess sodium dithionite (Na₂S₂O₄) in deoxygenated PBS buffer solution.

Scattering imaging and spectroscopy of single gold nanoparticles. The microscopy system consists of a Carl Zeiss Axiovert 200 inverted microscope (Carl Zeiss, Germany) equipped with a darkfield condenser (1.2<NA<1.4), a true-color digital camera (CoolSNAP cf, Roper Scientific, NJ), and a 300 mm focal-length and 300 grooves/mm monochromator (Acton Research, MA) with a 1024×256-pixel cooled spectrograph CCD camera (Roper Scientific, NJ). A few-micron-wide aperture was placed in front of the entrance slit of the monochromator to keep only a single nanoparticle in the region of interest at the grating dispersion direction. The true-color scattering images of gold nanoparticles were taken using a 40× objective lens (NA=0.8) and the true-color camera with a white light illumination from a 100 W halogen lamp. The scattering spectra of gold nanoparticles were taken using the same optics, but they were routed to the monochromator and spectrograph CCD. The immobilized nanoparticles were immersed in a drop of PBS buffer solution deoxygenated by clean nitrogen gas, the buffer liquid also served as the contact fluid for the dark-field condenser. The distance between the condenser and nanoparticles was 1-2 mm. The microscopy system was completely covered by a dark shield, which prevents ambient light interference and serious evaporation of the buffer solution.

Finite element simulation of electromagnetic energy coupling in PRET. For the simulations of the electromagnetic (EM) energy distribution presented in the text, we use a commercial software package FEMLAB available from Comsol Inc. (Los Angeles, Calif.) which numerically solves the Helmholtz equation for a set of predefined boundary conditions. The computation domain is a 1.2 μm×3.0 μm square with all sides treated as matched low-reflection boundaries. We set the ambient refractive index of the domain to be the value for water in accordance with the experimental setup. The excitation source is a plane wave with its electric field oscillating in the plane of propagation. Although the simulated wave from the excitation source experiences diffraction over its propagation, its wavefront approximates that of a plane wave in the length-scale of the nanoparticles under considerations. The refractive index of the gold nanoparticles is set to the values of bulk gold reported by Johnson and Christy^{[1]}. In order to cope with sharp resonance peaks, interpolated values of refractive index are used. Conjugated Cyt c molecule is simplified as a sphere, or a solid circle in 2D simulations. The real part of the refractive index of Cyt c molecules is assumed to 1.6 as most of other macromolecules. The imaginary part of the refractive index is calculated according the definition by Pope and Fry^{[2]}, n″ (λ)=∈λ/4π, where ∈ is the linear absorption coefficient of Cyt c and λ is the wavelength. Triangular elements are used for the computation mesh. We use the built-in mesh generator to regulate the mesh size in simulating different geometries. The distribution of local EM energy distribution is obtained from the built-in plotting function of FEMLAB and MATLAB.

A number of embodiments of the disclosure have been described. Nevertheless, it will be understood that various modifications may be made without departing from the spirit and scope of the disclosure. Accordingly, other embodiments are within the scope of the following claims.

1. A Plasmon resonance indicator comprising:
a metallic nanostructure that undergoes resonance when exposed to electromagnetic radiation; and
a binding ligand that binds to a mettalo-biomolecule.

2. (canceled)

3. A composition comprising a Plasmon resonance indicator of claim 1 in a pharmaceutically acceptable carrier.

4. A composition comprising a Plasmon resonance energy indicator of claim 1 linked to a metallo-biomolecule.

5. A method of detecting a metallo-biomolecule comprising:
exposing the composition of claim 4 to electromagnetic radiation, wherein the metallic nanostructure and metallo-biomolecule undergo Plasmon resonance energy transfer, and
detecting a spectral change when the metallic nanostructure and metallo-biomolecule are within resonance energy distance compared with either the metallo-biomolecule or metallic nanostructure alone.

6. The method of claim 5, wherein a distance between a nanostructure and metallo-biomolecule are changed.

7. The method of claim 6, wherein the distance is changed by cleaving a linking agent linking a nanostructure and metallo-biomolecule.

8. The method of claim 7, wherein the linking agent is a functional moiety on the nanostructure.

9. The method of claim 7, wherein the linking agent is a binding ligand.

10. The method of claim 9, wherein the binding ligand comprises a cleavable linker.

11. The method of claim 10, wherein the cleavable linker is a peptide.

12. A plasmon resonance indicator comprising: a nanoparticle that undergoes plasmon resonance upon exposure to an appropriate electromagnetic radiation, the nanoparticle having an analyte-binding region which binds an analyte an acceptor agent coupled to the analyte, wherein the nanoparticle and the acceptor agent are position relative to each other such that the nanoparticle and analyte undergo plasmon resonance energy transfer when the nanoparticle is contacted with electromagnetic radiation.

13. The Plasmon resonance indicator of claim 12, wherein the analyte comprises a metal.

14. The Plasmon resonance indicator of claim 12, wherein the acceptor agent is a metal.

15. The Plasmon resonance indicator of claim 12, wherein the analyte comprises a metallo-biomolecule.

16. A method for determining the concentration of an analyte in a sample comprising: contacting the sample with the plasmon resonance indicator of claim 12, exciting the nanoparticle; and determining the degree of plasmon resonance energy transfer in the sample corresponding to the concentration of the analyte in the sample.

17. The method of claim 16, wherein the step of determining the degree of plasmon resonance energy transfer in the sample comprises measuring resonance energy emitted from the acceptor agent of the analyte.

18. The method of claim 16, wherein determining the degree of plasmon resonance energy transfer in the sample comprises measuring resonance energy emitted from the nanoparticle, measuring resonance energy emitted from the acceptor agent, and calculating a ratio of the emitted energies from the nanoparticle and the acceptor agent.

19. The method of claim 16, wherein the step of determining the degree of plasmon resonance energy transfer in the sample comprises measuring the excited state lifetime of the nanoparticle.

20. The method of claim 16, further comprising the steps of determining the concentration of the analyte at a first time after contacting the sample with the nanoparticle, determining the concentration of the analyte at a second time after contacting the sample with the nanoparticle, and calculating the difference in the concentration of the analyte at the first time and the second time, whereby the difference in the concentration of the analyte in the sample reflects a change in concentration of the analyte present in the sample.

21. The method of claim 6, further comprising the step of contacting the sample with a compound between the first time and the second time, whereby a difference in the concentration of the analyte in the sample between the first time and the second time indicates that the compound alters the presence of the analyte.

23. The method of claim 21, wherein the compound is an inhibitor and the analyte is a metallo-protein.

24. The method of claim 23, wherein the metallo-protein is an metallo-enzyme.

25. (canceled)

26. A nanostructure that undergoes plasmon resonance energy transfer (PRET) when contacted with electromagnetic radiation.

27. The nanostructure of claim 26, comprising a geometric shell having an opening defined by a sharp edge.

28. The Plasmon resonance indicator or nanostructure of claim 26, wherein the nanostructure comprises one or more noble metals.

29. The Plasmon resonance indicator or nanostructure of claim 28, further comprising two or more layers of different metals.

30. The Plasmon resonance indicator or nanostructure of claim 28, further comprising functional groups attached thereto.

31. The Plasmon resonance indicator or nanostructure of claim 28, having optical properties.

32. The Plasmon resonance indicator or nanostructure of claim 28, having magnetic properties.

33. A Plasmon resonance indicator or nanostructure of claim 26, comprising a functional group that associates with a target analyte.

34. A method for detection of a target analyte, comprising:
a) providing a plurality of nanostructures;
b) contacting the plurality of nanostructures with a fluid suspected of or having the target analyte;
c) contacting the fluid with an electromagnetic radiation at a desired wavelength sufficient to cause plasmon resonance of the nanostructure; and
d) detecting plasmon resonance energy transfer (PRET) from a PRET partner in the fluid, wherein the PRET partner is indicative of the presence of the target analyte.
